(19) United States
(12) Patent Application Publication
(10) Pub. No.: US 2021/0110924 A1
(43) Pub. Date: Apr. 15, 2021

(54) SYSTEM AND METHOD FOR MONITORING SYSTEM COMPLIANCE WITH MEASURES TO IMPROVE SYSTEM HEALTH

(71) Applicant: AFFINITY RECOVERY MANAGEMENT SERVICES INC., Alexandria, VA (US)

(72) Inventors: Michael Tkach, Dayton, MN (US); Scott Storrer, Newtown Square, PA (US)

(21) Appl. No.: 17/066,942

(22) Filed: Oct. 9, 2020

Related U.S. Application Data
(60) Provisional application No. 62/912,762, filed on Oct. 9, 2019.

(51) Int. Cl. G16H 40/67; G16H 40/20; G16H 10/60; G16H 50/70; G16H 50/30; G16H 20/10; G16H 20/70; G16H 50/20; A61B 5/00; A61B 5/11; A61B 7/02

(52) U.S. Cl. CPC G16H 40/67; G16H 10/40; G16H 10/60; G16H 50/70; G16H 50/30; G16H 20/10; G16H 20/70; G16H 50/20; A61B 5/7275; A61B 5/1112; A61B 5/4809; A61B 5/486; A61B 5/4833; A61B 5/4803; A61B 7/02; G16H 40/20

(57) ABSTRACT

A system for monitoring and adjusting the psychiatric care of a human patient is disclosed. The system comprises a server, long-term memory storage, and a mobile computing device. When instructions are executed by the server, a feedback loop is established that causes: the mobile computing device to transmit data representing the human patient's actions; the server to retrieve from the long-term memory storage both baseline data related to the human patient and collective data related to a number of other human patients; the server to determine that the transmitted data either differs from the baseline data in a statistically significant manner or matches elements of the collective data in a manner that suggests a heightened risk of harm to the human patient; the server to transmit instructions causing a change in functionality of the mobile computing device; and the mobile computing device to implement the instructions.

### CROSS REFERENCE TO RELATED APPLICATION

This application is a non-provisional of the provisional application U.S. Pat. App. No. 62/912,762, filed Oct. 9, 2019, which is hereby incorporated by reference in its entirety.

### FIELD OF INVENTION

This application relates to systems and methods for evaluating and affecting the operation of another system via a feedback loop, and more specifically, to systems and methods for more directly influencing a patient's psychiatric care with the aid of a mobile computing device carried by the patient or computing devices made available to the patient's doctors and loved ones.

### BACKGROUND

In many complex systems, a malfunction may have catastrophic effect on the system as a whole, and yet warning signs may be difficult to detect until a moment at which the system is already in an unrecoverable state. This can be true of mechanical devices (such as an airplane where a small stress fracture in a wing can quickly develop into a full shearing of the metal under the stresses of flight), biological systems (such as a human individual who, while in treatment for addiction to a dangerous substance, may be triggered by a stimulus or have a sudden impulse to take a dose of the substance and cause relapse or overdose), or even interconnected systems of persons and/or mechanical devices (such as a corporation where best practices are not followed and legal liabilities are incurred because corporate actors did not address a situation as they should have, or a computer network where performance is degrading due to a denial of service attack or the breaking of one or more communication links between nodes).

Thus, there is a need for better predictive analytics and preventative action to prevent catastrophic failure that could have been prevented with targeted action in response to the information known before the point at which the catastrophe could no longer be avoided.

### BRIEF SUMMARY

Described herein are methods and systems for monitoring compliance with preventative or remediative measures in a system, for determining the effectiveness of variations in those preventative or remediative measures through iterative tracking of objective and subjective inputs from a system, for establishing feedback loops to iteratively modify system behavior based on received input, and for anticipating a potential system failure before it occurs in order to mitigate or entirely prevent it.

In particular, a system for monitoring and adjusting the psychiatric care of a human patient is disclosed. The system comprises a server, long-term memory storage, and a mobile computing device, and the server and the mobile computing device comprise instructions that, when executed by a processor of the server, establish a feedback loop. The feedback loop causes the mobile computing device to transmit data from one or more sensors of the mobile computing device representing the human patient's actions while in possession of the mobile computing device; the server to receive the transmitted data; the server to retrieve from the long-term memory storage both baseline data related to the human patient and collective data related to a number of other human patients; the server to determine that the transmitted data either differs from the baseline data in a statistically significant manner or matches elements of the collective data in a manner that suggests a heightened risk of harm to the human patient; the server to transmit, to the mobile computing device, instructions causing a change in functionality of the mobile computing device; and the mobile computing device to implement the change in functionality upon receipt of the instructions.

A computer-implemented method for monitoring and adjusting the psychiatric care of a human patient is disclosed. The method comprises establishing a feedback loop that comprises receiving data transmitted from one or more sensors of a mobile computing device representing the human patient's actions while in possession of the mobile computing device; retrieving from long-term memory storage both baseline data related to the human patient and collective data related to a number of other human patients; determining that the transmitted data either differs from the baseline data in a statistically significant manner or matches elements of the collective data in a manner that suggests a heightened risk of harm to the human patient; and transmitting, to the mobile computing device, instructions causing a change in functionality of the mobile computing device.

### BRIEF DESCRIPTION OF THE DRAWINGS

FIG. 1 depicts, in simplified form, an example interconnected system of devices capable of performing methods described within the present disclosure;

FIG. 2 depicts, in simplified form, a feedback loop implemented by an automated risk assessment system according to the present disclosure;

FIG. 3 depicts, in simplified form, a central computing device in communication with several monitored systems and using received data to compare performance among them;

FIG. 4 depicts an example tree structure for use within a random forest algorithm for risk assessment, according to the present disclosure;

FIG. 5 depicts one example method of classifying and reclassifying physical locations based on a combination of received objective geolocation data and subjective reporting on patients' actions; and

FIG. 6 depicts, in simplified form, a feedback loop implemented by an automated risk assessment system taking into account how a system varies in behavior both from the system's own baseline and from other systems similarly situated.

### DETAILED DESCRIPTION

To address the potential errors and unpredictability inherent in human supervision of and maintenance of a system whose functioning must be monitored, the present disclosure describes an automated system that receives data from a variety of sources regarding the monitored system, automatically selects possible responses to the received data that are determined to be most likely to improve or maintain system function, and create a positive feedback loop as the monitored system is acted upon, causes new data to be produced for analysis, and additional responses are automatically selected.

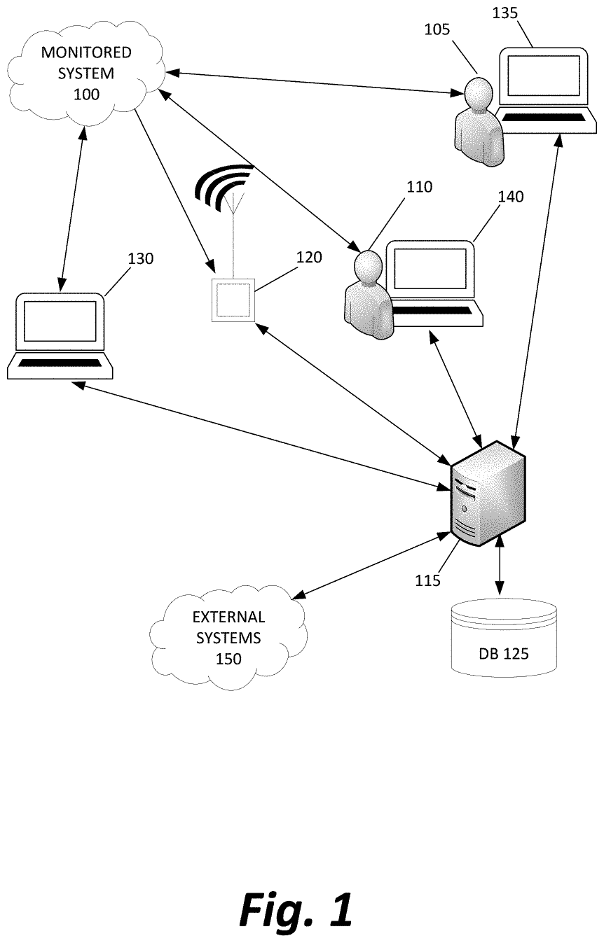

FIG. 1 depicts, in simplified form, an example of a system 100 that is in indirect communication with and is monitored by a central computing device 115.

The central computing device 115 monitors the system 100 for compliance via one or more electronic sensors 120 that are either standalone sensors in communication with the central computing device 115 or are built into computing devices in communication with the central computing device 115 and that report gathered sensor data over that channel of communication.

The central computing device 115 also monitors the system 100 via various intermediate computing devices 130, 135, and 140, used by the system 100, stakeholders 105, and experts 110, respectively. Such intermediate computing devices may be, for example, laptops, desktops, tablets, mobile phones, or any other generic computing device allowing entry of data and communication via a user interface.

The central computing device 115 receives a number of objective data values from the electronic sensors, and receives both objective and subjective data values from the intermediary computing devices 130, 135, and/or 140. The central computing device 115 then uses the received data values to shape a feedback loop that induces the monitored system 100 to improve its performance, according to the objective and subjective data, and avoid various modes of system failure.

Objective inputs to the central computing device 115 represent data that is directly and quantitatively measured by an automated device or by mathematical processing of or retrieval of data that has been measured in the past. In contrast, subjective inputs to the central computing device 115 represent data that may be either quantitative or qualitative, but most importantly is based on a human evaluation of a situation to classify or describe an attribute witnessed by the human.

The objective inputs to the central computing device 115 from the monitored system 100 include sensor data from the electronic sensors 120 on or within the monitored system 100, or reports from sensors with which the monitored system 100 interacts, such that no human judgment of the situation is exercised. Instead, numbers or true/false values are automatically generated that quantify some measurable attribute of the monitored system 100. These include, in various implementations, some subcombination of geolocation data (e.g., from a global positioning system (GPS) sensor, from proximity to and transmission of data via a cell tower in a known location or from triangulation based on data connections to multiple cell towers, from near field communication or radio frequency identification (RFID) tag interaction with an item in a known location, or from other contextual clues, such as an IP address being used by a device to access the Internet being associated only with an internet service provider in a particular location or region), accelerometer data, data from other physical sensors (e.g., thermometer, microphone, camera or other optical sensors, motion sensors, barometer, etc.), data gathered by laboratory equipment or medical devices (e.g., spectrometer, gene sequencer, sensor for detecting presence of a particular chemical in a sample, breathalyzer, O₂ sensor, sphygmomanometer, etc.), log data from computing devices (e.g., when a device was in use, when a file was received, accessed, or transmitted, other usage reports or statistics, what features or functions of a device were in use at a particular time, etc.), or any other source of data that returns a concrete value for an attribute of the monitored system 100 (i.e., it does not rely on inferential analysis by a computer, or by a human making a judgment call regarding how to classify the attribute).

Subjective inputs, in contrast, include survey or polling data from: within the monitored system 100 itself (i.e., participants within a system, a medical patient himself, or the employees of a corporation), from one or more stakeholders 105 in the success of monitored system 100 (e.g., users of a system external to the system, family members of a patient, or customers or owners of a corporation) and who observe at least some of monitored system 100's functioning, one or more subject matter experts 110 (e.g., information technology professionals, doctors or counselors treating a patient, or consultants hired by a corporation) who are examining the monitored system 100, or even output from artificial intelligence subsystems that use fuzzy logic or non-deterministic methods to give a classification or other assessment of provided inputs. Subjective inputs are gathered from the stakeholders 105 and subject matter experts 110 using their computing devices 135 and 140 to access a web application, mobile app, or other graphical user interface and enter data regarding the monitored system 100 to be conveyed to and processed by the central computing device 115. Subjective inputs are also entered, if the monitored system 100 includes or is a human being, by that human being into a graphical user interface of the human's computing device 130.

Data received from the monitored system 100 (and from other monitored systems similar to the monitored system 100 and to which the monitored system 100 may be compared) is stored in one or more databases 125 communicatively coupled to the central computing device 115.

The central computing device 115 is also in communication with a number of external systems/gateways/APIs 150 through which it can obtain information on monitored system 100 or automatically cause an interaction to occur with monitored system 100. Examples of such external systems include an email server, an SMS gateway or other API for sending text messages, fax machine, financial computing systems such as banks or payment providers, and interfaces with laboratories to obtain lab results. The communication between the central computing device 115 and external systems 150 is in some cases performed through an XML/SOAP (Extensible Markup Language/Simple Object Access Protocol) API to allow sending and receipt of complex, structured data objects, though simpler protocols may be used when performing a straightforward task such as causing a text message to be sent to a given telephone number, and alternative protocols may be used as suitable for any given function.

All of the connections for input and output—with the various devices and gateways 120, 130, 135, 140, and 150—are used to accomplish a feedback loop upon the monitored system 100.

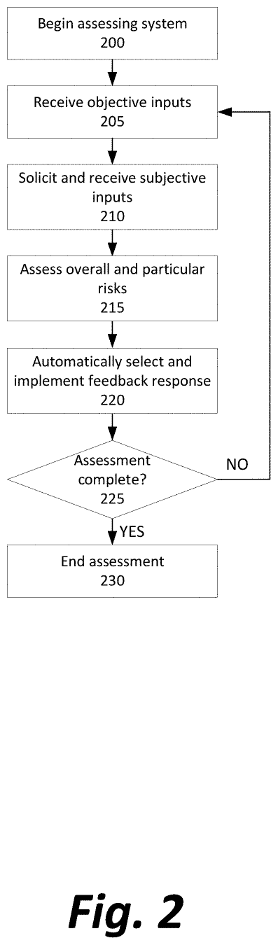

FIG. 2 depicts, in simplified form, a feedback loop implemented by an automated risk assessment system—for example, a system comprising the central computing device 115 illustrated in FIG. 1 and whose actions are performed by a computer processor in the central computing device 115 executing instructions on that same device—that acts to preserve the proper function of the monitored system 100.

The objective and subjective input values are initially assessed (Step 200) to determine a baseline set of values. The objective values are obtained (Step 205) and the subjective values are solicited from those who are able to provide them, then iteratively assessed over a series of periods of time (Steps 205 and 210).

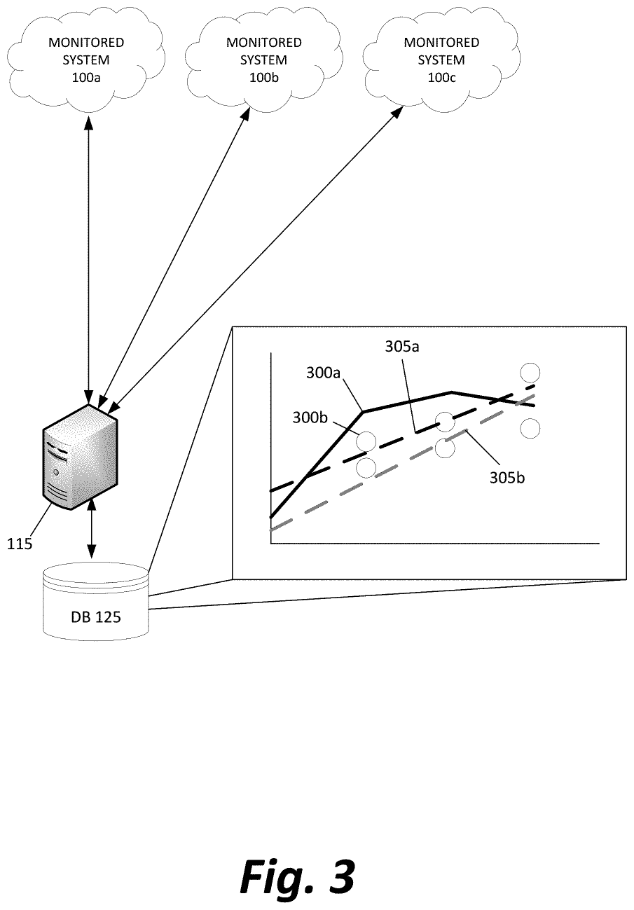

At each successive assessment, the obtained objective and subjective input values are each compared (Step 215) not only against the particular system's baseline for that value (i.e., “Has this system attribute improved since the assessment began?”) but also against a set of previous assessments of that value (i.e., “Is the trendline for this system attribute currently positive or negative?”), if available. The comparison determines the existence of and severity of any risk to the system based on downward trajectory in one or more attributes or similarity to known monitored systems in the past that have thereafter faced difficulty or malfunction. In some implementations, a considerable number of similar monitored systems are concurrently assessed or have been assessed in the past to aid in making this determination. (FIG. 3 depicts such an implementation and is described in further detail below).

The risk assessment will, after each assessment during an interval of time, trigger an automated response in the form of one or more commands to a computing device capable of receiving the command and acting upon it without human intervention (Step 220). Example commands include a command to an alarm to activate, a command to automatically trigger an application or procedure on a remote computing device, or a command to a visual display to begin displaying a notification to a human actor, regardless of whether the human actor intended to check for or access the notification. Other actions may be automatically taken during each interval in order to provide feedback and improve the functioning of monitored system as measured by the objective and subjective inputs. For example, if a particular attribute value is abnormally low or has a poorer trendline than others among the values assessed in the monitored system 100, a targeted automated action is triggered to cause a change within the monitored system 100 in hopes of improving the particular assessed attribute value over time.

In some implementations, the automated response includes generation of a report that can be accessed by a human actor through a graphical user interface, such as a web browser, and which the human actor can take into consideration in formulating a plan of action.

Advantageously, using the approaches described herein, data relationships among variables that were not previously known can be discovered by processing data in aggregate from a number of similar monitored systems 100 being assessed. It may be discovered, for example, that an independent variable thought to have a high determining effect on a system outcome actually has very little correlation with the outcome, or in contrast, that a tracked variable that is initially thought to be unimportant actually has a very strong predictive ability. In one implementation, a random forest structure is used to assess a probabilistic risk of system failure based on combinations of the received objective and subjective input values.

Outputs from the system include messages to one or more of: the monitored system 100 itself (such as commands to an interface of an electronic device, or instructions to be followed by a human), the stakeholders 105 (informing them of the current status of the system and of actions they can take to mitigate or prevent system malfunction), or the subject matter experts 110 (indicating that an intervention requiring the expert's expertise is advised).

Until it is determined that assessment of a particular system is complete (Step 225), automated actions are continually part of a feedback loop. Within the loop, after taking action that affects monitored system 100, more data is gathered to determine the course of action for a following interval of time (back to Step 205 and following). When the loop is to be completed and monitoring of the system 100 is complete, any outcome data is stored to allow the prediction of outcomes of future monitored systems based on their similarity during the monitoring process to the previously monitored system 100.

As mentioned before, FIG. 3 depicts, in simplified form, a central computing device 115 in communication with several monitored systems 100 and using received data to compare performance among them.

The monitoring of many systems 100 allow the generation of a database of current and historical system behaviors that allows the objective or subjective values 300a in any particular system 100a to be compared to the values 300b of other systems 100b-100n (i.e., “Is the current value of a given attribute high or low within the range seen in similar systems, or high or low compared to the average value of that attribute in other systems?”) and for trends 305a in one or more values of the particular system 100a to be compared to trends 305b in the values of other systems 100b-100n (i.e., “Are the trendlines for an attribute in other systems more positive or more negative than the trendline for this attribute in this system?”).

When the monitored system 100a is compared to other, similar systems 100b-100n, additional comparisons are made against the values each similar monitored system 100b-100n had when at a similar stage of assessment (e.g., comparing the present values of a monitored system that began to be assessed two weeks ago to values from a monitored system that had begun assessment two weeks before, even though that assessment had occurred a year ago) or at the present time, regardless of how long other systems 100b-100n have been assessed (e.g., comparison in the present between one monitored system that began assessment one week ago, and another monitored system that began assessment three weeks ago). Over time, a massive number of trendlines 305a and 305b are gathered, for a number of monitored systems 100 and for a number of objective and subjective values 300 for each of those monitored systems.

Assessment of these values or trends as a cause for concern and intervention (whether in an absolute sense or relative to other monitored systems 100b-100n) is initially performed, in one implementation, by grouping similar input types and processing through a random forest algorithm. Outputs from one forest of trees related to a particular aspect of system functionality act as inputs to other forests of trees, cascading from initial input values of system attributes to intermediate nodes that may indicate or classify particular risk features or modes of failure, and from those intermediate nodes to a final node that indicates or classifies an overall systemic risk. Intermediate and root nodes store one of a number representing a probability of a particular failure mode, a selection of a particular most important aspect of the system to address, a yes/no determination regarding whether an elevated risk exists overall or within a particular aspect, a classification of a particular risk factor as urgent/moderate/mild/not applicable, or some other qualitative or quantitative measure that can be compared to a threshold for action.

Based on the above identification of a danger or highest priority (for example, as a result of one or more of downward trends in one or more of the assessed values over time, abnormally low values for one or more system attributes compared to other systems' attributes and regardless of trend, or similarity of the assessed values to those of another historical system that later suffered a malfunction), a risk assessment is automatically generated indicating that some form of intervention is needed or desirable.

The preceding generic system and method may be adapted into a variety of specific implementations that are tailored to particular problems in such diverse fields as manufacturing, medicine, and computer networks.

A particular application is found in the context of a centralized system that tracks a number of human patients (each corresponding to the monitored system 100) who are undergoing treatment for substance abuse and that intervenes in various ways independent of or in parallel to a medical treatment team to improve patient outcomes by replacing “one-size-fits-all” treatment plans with an approach responsive to the needs of each particular patient. The centralized system does not automate current practices, but instead supplements existing care with an automated healthcare system that is more reliable, less error-prone, and more adaptive.

As described in a more general sense in previous paragraphs, an automated healthcare management system according to the present disclosure takes in a variety of objective and subjective inputs regarding a human patient (including, among other factors, the patient's current mental health, cravings, risk of relapse or self-harm, and overall compliance with treatment) to determine the risk to a patient of relapse or self-harm at any given moment in time, track the patient's overall progress during treatment, create positive feedback loops based on the data received at each stage of treatment, and intervene to prevent a negative feedback loop from forming and the patient self-harming or relapsing before a treatment team of doctors, counselors, or others can prevent the harm.

Objective Inputs

When a patient begins to be tracked by the system, the patient may undergo an initial genetic testing/sequencing to predict reactions to medication, predict behavior, and/or to enable correlating genes that currently have unknown qualities with behavior or reactions seen in a number of patients over time.

Other information entered at the commencement of tracking includes one or more of age, gender, medical history, and current diagnosis, or other less medical and more socioeconomic data such as address, income, occupation, marital status, presence of children, education level, etc.

At numerous times during tracking by the system, the patient undergoes drug testing via analysis of the patient's urine, blood, saliva, hair, nails, or breath (breathalyzer). The testing is performed at a trusted laboratory in communication with the system or possibly via equipment available to the patient himself (such as a breathalyzer built into the patient's car or capable of being connected to the patient's mobile phone as an accessory). The samples from the patent are tested for any indication that forbidden substances have been used (such as the presence of, or metabolites of, an opioid or other chemical to which the patient is addicted). The system also receives and tracks a list of prescribed medications for the user, and the patient is tested for the presence of expected substances (such as metabolites of a medication) that verify that the patient is taking all prescribed medications.

Verification that the patient is cooperating with a regimen of medication is determined by the presence of indicators that the patient has used one or more of: opioid addiction medications (including buprenorphine (Suboxone®), methadone, or naltrexone (Vivitrol®)), alcohol addiction medications (including acamprostate (Campral®), disulfiram (Antabuse®), or naltrexone), nicotine addiction medications (including bupropion (Wellbutrin®) or varenicline) or nicotine replacement therapy (nicotine gum, etc.), antidepressants (including trazodone (Desyrel®, Oleptro®), fluoxetine (Prozac®), bupropion, sertraline (Zoloft®), paroxetine (Paxil®), venlafaxine (Effexor®), Escitalopram (Lexapro®), Duloxetine (Cymbolta®), Atomoxetine (Strattera®)), medications for bipolar disorder (including lithium carbonate, olanzapine/fluoxetine, carbamazepine (Tegretol®)), anti-obsessional medications (including fluoxetine, sertraline, paroxetine, fluvoxamine (Luvox®), citalopram (Celexa®), escitalopram (Lexapro®)), psycho-simulants (including methylphenidate (Ritalin®, Concerta®), dexmethylphenidate (Focalin®), or lisdexamphetamine (Vyvanse®)), antipsychotics (including quetiapine (Seroquel®), loxapine (Loxitane®), trifluoperazine (Stelazine®), risperidone (Risperdal®), lurasidone (Latuda®), aripiprazole (Abilify®)), and anti-anxiety medications (diazepam (Valium®), clonazepam (Klonopin®), lorazepam (Ativan®), alprazolam (Xanax®), or buspirone (BuSpar®)).

Other objective inputs to a monitoring system include those related to the patient's location. A mobile computing device or wearable computing device of the patient provides geolocation data on the patient's movements, whether through a global positioning system (GPS) sensor, data based on cell towers to which a mobile device has connected, or radio frequency identification (RFID) tags associated with a location and that a computing device has been close enough to detect. In some implementations, a patient's physical exercise is estimated based on distance traveled, and the patient's physical location or presence at particular predetermined locations may be known to the system.

If the patient regularly possesses or holds a mobile computing device or wearable computing device, it provides accelerometer data that enables tracking of a patient's physical movement. This information is used, among other applications, to infer whether the patient is sleeping or active at a given time, and if sleeping, the quality or deepness of the sleep.

Other data is provided from a mobile phone or wearable device, based on sensors present in the device (camera, microphone, etc.) or based on the use of the device itself (whether the device has been used to view content, whether the device is turned on, what proportion of the device's use has been devoted to treatment related matters and unrelated matters, whether and how much the device is being used for communication as opposed to consumption of content, etc.). For example, a microphone can be used to record the user's voice for indicators of stress or anger based on tone, volume, or words used. The camera may be used to take a picture of a prescription bottle and automatically populate data fields related to the prescription (e.g., ordering physician, name of drug, quantity included, and dose instructions and amounts) via text recognition. Additionally, a camera can be used to capture signatures verifying attendance for meetings or counseling sessions. A picture of a signed attendance card is uploaded and the system identifies the presence or absence of a signature to verify meeting attendance.

Subjective Inputs

A graphical user interface (GUI) is provided, and is delivered by one or more of a web page viewed with a web browser, a dedicated mobile app, and any other presentation format. The GUI is generated to allow the patient, family members or other contacts, and doctors or counselors to enter survey data.

Survey questions presented by the GUI might be keyed to a response index—for example, from 1 to 10—and include “How happy are you?”, “How stressed do you feel?”, “How afraid are you of a relapse?”, and so on. For users who are not the patient, the questions are phrased to elicit how they believe the patient is feeling based upon their personal knowledge and interactions with the patient, and/or elicit how the non-patient user is feeling, as both pieces of data may be important to determining the patient's mental health from the patient's interactions with the non-patient user or the patient's interactions with other persons that were witnessed by the non-patient user.

Other questions are of a nature that require an answer not keyed to a scale, but are still numerical or binary. Such questions might include “Did you take all prescribed medications today?”, “How many hours did you sleep last night?”, or “Are you experiencing any desire to self-harm?”.

Further questions are more open-ended, including prompting a user to record, in free-form, their current mental state, the content of their dreams, or other information that might be considered by a psychologist in treatment of the patient and understanding the patient's current mental state.

Feedback Cycle

Using all of the above data, patient health is assessed in light of the American Society of Addiction Medicine's six domains:

Detox/Withdrawal Potential/Risk: These factors are assessed by, by way of non-limiting example, self-reporting of cravings and sobriety and reporting of others on patient sobriety, as well as the objective measures of drug screening or breathalyzer results.

Biomedical Conditions: These factors are assessed based on, by way of non-limiting example, medical history, genetic testing, physical exercise, or other factors indicating the current physical health of the patient.

Emotional & Behavioral Conditions: These factors are assessed based on, by way of non-limiting example, the patient's own survey data, survey data from family interacting with the patient, and survey data from therapists or doctors who have consulted with the patient.

Readiness to Change: These factors are assessed based on, by way of non-limiting example, compliance with suggested activities, such as viewing suggested educational content, attending support meetings, exercising or performing other goals, and interaction with the treatment team as described in survey data.

Relapse, Continued Use, or Continued Problem Potential: These factors are assessed based on, by way of non-limiting example, drug screening (both for absence of harmful substances and presence of prescribed medications), self-reporting surveys, and surveys of family and doctors. The survey data may be direct, such as asking whether the patient is sober, or indirect, such as asking whether the patient is or appears to be struggling with cravings, depression, quality of life issues, etc., that may lead to a relapse in the future.

Recovery Environment: These factors are assessed based on, by way of non-limiting example, survey data from family members and interaction between family members and a treatment team. Risk is downgraded or upgraded in the system's estimation based on whether family appear to be cooperating both with the patient and with the treatment team, and whether the household environment is conducive to recovery or instead is introducing additional stresses that make recovery less likely.

As progress is tracked within each of these six domains, automated actions are taken by the risk assessment system with respect to the patient. The treatment team receives reports output by the centralized system so they are better informed regarding how to target treatment within their domains, the patient is encouraged and plays a greater role in their own treatment, and family of the patient can better support the patient and aid in the treatment process.

The automated risk assessment is performed within the centralized system (i.e., by a processor of central computing device 115 or another computing device in communication with it) and uses a variety of decision trees arranged as a random forest to provide a classification of a current risk level or issue.

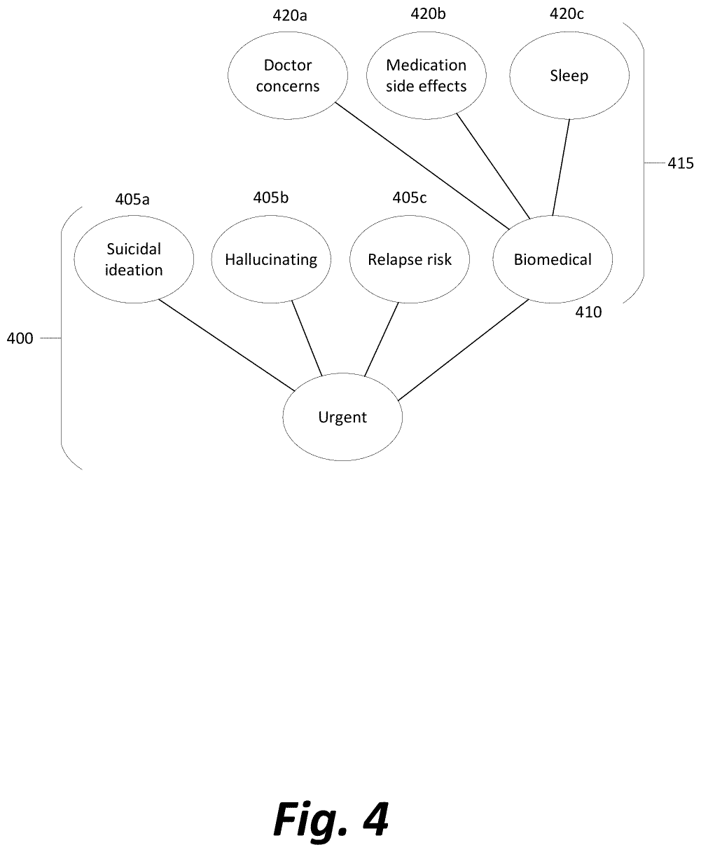

FIG. 4 depicts, in simplified form, an example decision tree structure for use within a random forest algorithm for risk classification.

As shown in the example FIG. 4, an “urgent issue” tree 400 may have, as leaf nodes 405a-405n, gathered data on various risk factors, such as whether the patient has recently experienced suicidal ideation, whether the patient has recently experienced mental hallucinations and whether those hallucinations included an urge to harm others, whether the patient is classified as an urgent relapse risk for substance abuse, and whether the patient is experiencing an urgent biomedical issue.

Some leaf nodes 410, such as the urgent biomedical issue mentioned above, act more as intermediate nodes, in that they are not only a leaf node of a main tree 400, but are themselves the root node or output of another decision tree 415. In this example, the secondary tree 415, representing whether an urgent biomedical situation exists, has as its own input leaves 420a-420n whether there are noted medical concerns by a professional, whether the patient is experiencing side effects from medication, and whether the patient has had more than a predetermined amount of sleep during a recent window of time.

Other values that are leaf nodes 405 or 420 or intermediate nodes 410 in an overall risk determination include, for example, overall assessment nodes for relapse risk, mental health, and physical health; self- or other-reporting of patient trauma; self- or other-reporting of patient depression; self- or other-reporting of patient anxiety; self-reporting, reporting by others, or laboratory analysis that indicates patient use of opioids, alcohol, or stimulants; the patient's past or present experience of chronic back pain, migraines, or other pain; presence of disturbing nightmare imagery in the patient's dreams, drug use in dreams, or other dream content; the duration, regularity, quality, or other aspects of the patient's sleeping patterns; the quality of patient interaction with family and family support for treatment; whether the patient is employed; other positive or negative social or environmental factors experienced by the patient; whether the patient is attending counseling, support meetings, appointments, or other external support structures; and the patient's age, gender, marital status, race, income, or other demographic factors.

A forest's overall vote is determined based on the majority vote of the trees within the random forest, or may instead be weighted such that only a predetermined proportion of the trees less than 50% need to indicate an elevated risk before action is taken. If the forest overall vote results in an output of “yes” (or “elevated” or “urgent” or “danger”, etc.), an automated response is triggered. In cases where a mild issue is highlighted, the response could include the automatic transmission of educational content to be shown on the patient's computing device, providing the patient with better coping methods for the issue being faced. Where a more serious issue is detected, the automatic response could include generation of a report to emergency health services including the patient's geolocation data and a report to personal devices or web interfaces accessible by the patient's family and treatment team, apprising them of the risk.

The random forest's trees are, in one implementation, initially modeled after the structure of existing decision trees in questionnaires such as the Patient Health Questionnaire-9 (PHQ-9) that are known in the medical field. However, the trees can then be varied to assign different weightings to each factor, eliminate factors from consideration, or rearrange the relationships between factors in a decision tree and the risk predicted can be compared to the actual outcome for a patient, providing data on whether a particular tree variant was more or less effective in risk classification than tree variants previously considered.

As larger data sets are built up, deep learning techniques may further be used to automatically determine whether diagnostic or survey data gathered by a treatment team has greater significance than was initially thought, or, instead, has less or no statistical significance on patient outcomes when compared with the intuition of the treatment team. Deep learning techniques may also be used to automatically refine risk determinations and compare possible treatment plans by efficacy or overall risk.

During the feedback cycle, information can also be gathered that only indirectly relates to any given patient but is still useful to treatment providers and assessment of patient actions.

For example, the central computing device 115 maintains a searchable database of support group locations and meeting times to allow patients to find a group to attend. Even if a given location and time is not confirmed in any database of support group meeting locations, it may be confirmed based on data received during the feedback cycle over a period of weeks.

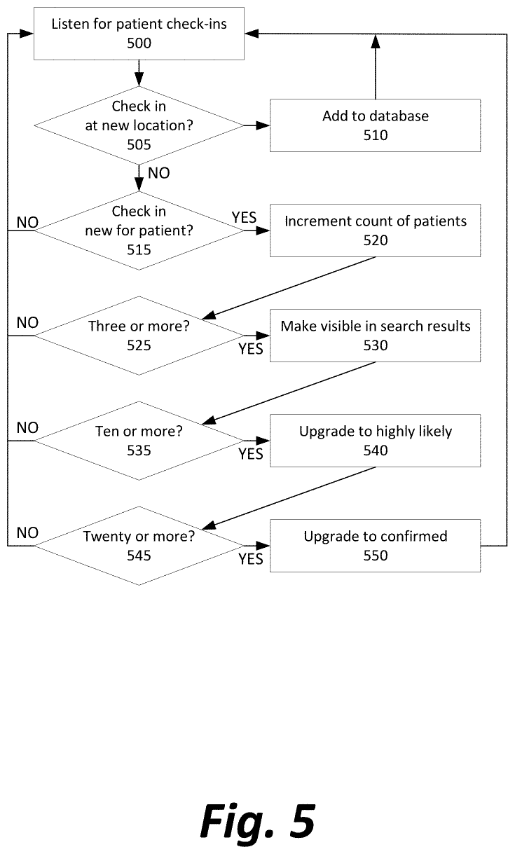

FIG. 5 depicts, in simplified form, a method of classifying and reclassifying physical locations based on a combination of received objective geolocation data and subjective reporting on patients' actions.

The central computing device 115 is always listening (Step 500) for messages indicating that a patient has attended a support group meeting, using software on patient computing devices 130.

A patient undergoing treatment may, at any time, indicate that they are currently checking into a support meeting, or recently attended a support meeting that is currently unknown in the database of meeting locations and times (Step 505). The location may be self-reported by the patient as an address, may be entered by the patient with the help of a mapping application or widget, or, in one implementation, is automatically taken from current geolocation data provided by a mobile or wearable computing device of the patient. The scheduled time and duration of the meeting are also determined, either automatically, based on the timestamps of the arrival and departure of the patient at the location each day or each week, or based on data entered by the patient. The patient also indicates the type of the meeting, whether the meeting is open, closed, or private, assign the meeting a rating on a one-to-five-star scale or other scale. The gathered information is used in order to aid other patients in searching for a possible meeting to attend in their area, whether in an area the patient usually resides or an area that the patient is temporarily visiting and is less familiar with.

The location is entered into a database (Step 510) to be stored along with a number of check-ins initially set to one.

If the location and time are already known, but that particular patient has never been there before (Step 515), the record in the database is updated (Step 520) to increment the number of patients who have confirmed attending that meeting.

By default, meeting locations in the database are not searchable or discoverable. If, however, three or more patients have confirmed attending a meeting (Step 525), it is toggled (Step 530) to a discoverable state that patients can find by searching for meetings nearby.

Once ten patients have confirmed attendance at a meeting (Step 535), it is shown in search results as a “highly likely” meeting (Step 540).

Once 20 patients have confirmed attendance at a meeting (Step 545), it is upgraded to a “confirmed” meeting (Step 550).

In parallel to the above process, the central computing device 115 also periodically checks to see if any meetings stored in the database have not had a check-in for an extended period of time, and if so, to downgrade the certainty that a meeting is expected to occur. For example, even if a meeting is currently set to “Confirmed,” if there are no check-ins at the given place and time for a period of 30 days or more, it should be downgraded to only a “highly likely” meeting. If any “highly-likely” meeting (including one that was just downgraded) has no check-ins for a further 30 days, it should be downgraded to a merely-discoverable meeting, and upon another 30 days without check-in at a discoverable meeting, it should be toggled to undiscoverable. Thus, over a period of up to 90 days, any locations that are not in use will be cleared out from the database. Downgraded meeting locations have their records updated to indicate that a fixed number of additional check-ins by new patients will be needed before they can be upgraded again (for example, needing at least seven new patients to go back to “highly likely” status, and at least ten new patients to go back to “confirmed” status).

The particular numbers of people, intervals of time, stages of classification, etc., described above are provided purely as an example implementation, and differing thresholds or classifications may be used to achieve a different tolerance for uncertainty or speculation in search results or a different manner of describing search results to a patient.

Patients are also able to check-in to known locations, such as those imported from a listing of businesses (especially doctors and therapists) in an area, or locations already entered or verified by a system administrator.

In addition to tracking the patient's self-reporting of group meeting attendance, the monitoring system also confirms the patient's attendance through geolocation data. If a patient leaves a group meeting before its scheduled duration has elapsed, or only stops briefly to check in and keeps moving thereafter, the system will record the apparent non-compliance and may generate an alert prompting the patient to return to the meeting or explain the departure, and provide this information to the treatment team in order to help them assess the patient's willingness to participate in treatment.

Outputs

Outputs/actions of the automated healthcare management system include, in one example, automatically sending contextual messages or feedback to the patient. In some implementations, these messages are as simple as showing the progress by the patient in metrics being tracked by the system, and sending messages of encouragement to the patient.

In other implementations, the system automatically presents, or prompts the patient to view, an article or video in order to educate the patient and provide them with new ways of viewing a situation, new tactics for addressing a situation, and so on. The system tracks what content has been viewed and the patient's rating of the content, in order to confirm that the patient is participating, avoid showing the same content repeatedly, and fine tune content suggestions that are generated by the system to provide similar content to that which has been deemed helpful by the patient. The system also tracks the patient's current mental state in order to provide the most topical content for dealing with a present issue. A cascading priority of possible content types is used; for example, any patient currently experiencing suicidal ideation will be shown content related to preventing suicide, any patient who is not suicidal but is experiencing substance cravings will be shown content related to overcoming those cravings, and those facing less-serious issues will be shown content related to those issues if and only if all other, more pressing issues have been resolved. In one implementation, issues are prioritized as follows: suicidal ideation, relapse, urgent mental health issues, urgent biomedical issues, issues related to support system, general medical issues, general sleep and wellness issues, and issues related to a patient's demographic data (age, gender, race, sexual orientation, or employment). If none of these issues exist, completely generic self-help content may be provided.

A similar cascading priority of responses may occur after a patient has used a mobile app or web interface to fill out a standardized psychological questionnaire (such as the Columbia-Suicide Severity Rating Scale). For example, if a patient initially answers “no” to three questions related to depression and suicidality, the questionnaire simply terminates with no particular action by the system in that moment. However, a “yes” to any of those questions will trigger an automatic notification to at least one member of the health care team, and “yes” on additional follow-up questions can also trigger messages to family members or other local support for the patient or to emergency services. Similar cascading responses are also set up as automatic reactions to the patient's answers in other standardized questionnaires, such as the PHQ-9, Generalized Anxiety Disorder-7 (GAD-7), alcohol craving screener, opioid craving screener, substance craving screener, sleep quality screener, dream content screener, impulsivity screener, anger screener, and medication adherence screener.

For another example, the system may require the patient to undertake another particular task or goal, such as self-performance of cognitive behavioral therapy, attending a group meeting, or going out for a walk or exercise. The system then awaits either the patient's self-reporting that the task has been completed, verification that the task has been completed by another individual who observed or participated in the completion of the task, or automatically based on information derived from sensors on a mobile computing device or other computing device.

For another example, the system generates messages based on a sensed geolocation of the patient through the patient's mobile phone or other device worn on the patient's person. When the patient is in a new location, like a city different from the patient's home city, the system uses the previously described database of confirmed or likely support group meetings to generate and display messages indicating where a nearby support meeting may be held during an upcoming time window. If the patient attends a support meeting but leaves before the meeting is scheduled to end, the system displays a message prompting the user to return and remind the user that compliance with a requirement to attend a meeting will be recorded.

For another example, the system may generate messages or automated actions based on accelerometer data or other data from a mobile computing device or wearable computing device, showing whether the patient is likely to be asleep, and if so, for how long. If a patient does not appear to be asleep during a time of the night that the patient should be sleeping for maximum restfulness and mental health, the system causes the patient's mobile phone or other computing device to display a message prompting the user to stop using the device and go to sleep. The mobile device may also be automatically configured to act in ways conducive to sleep, such as changing screen characteristics to limit blue light output or overall brightness, changing notification settings to minimize distraction, decreasing device output volume, automatically disabling an internet connection, cellular data connection, or other connection of the device, automatically closing or opening an application on the device, automatically powering down the device, automatically playing white noise or other sounds conducive to sleep, and so on. Geolocation data may also be used to augment automatic sleep improving techniques, for example by having white noise or nature-related sounds increase in volume if a thunderstorm is near the patient, or by relating the timing of actions to sunset or sunrise for a more-natural timing of the patient's falling asleep or waking up.

The system also automatically sends any of a variety of messages to family or other contacts of the patient. These messages include one or more of tracking of the patient's progress, certification that the patient is remaining sober, a prompt that the patient may be at risk and need particular support or monitoring, a warning that the patient is no longer sober, or display of educational content that helps the person to support and interact with the patient.

The system also automatically sends any of a variety of messages to doctors or counselors who are part of the patient's treatment team. The messages include raw data from the objective and subjective data sources, compilations/summaries/digests of that data and trends over time, or suggestions that a patient's risk is decreasing or that it is increasing and greater intervention may be needed.

In some instances, the system assesses that a patient's risk is high enough that it is prudent to send a message to emergency services, indicating that a patient may be an imminent risk of self-harm or overdose. The message is automatically sent using, for example, a call with Voice over IP (VOIP) and a synthesized voice or pre-recorded message, a mobile phone's text message to an emergency services number, or some other protocol capable of being received and promptly processed by emergency services.

A graphical user interface generated by the system organizes all the received data so that the patient, family, or doctors are able to see (in tabular, chart, or other visual form) trendlines, see data from a given day or window of time, see all data from a given data source, or otherwise access some or all objective and subjective data received by the system throughout the period of treatment.

In some implementations, the system also facilitates HIPAA-compliant audiovisual communication between the patient and a doctor to allow for more fruitful consultation without the patient and doctor having to be physically present in the same location. Other communication channels are included in other implementations, including audio-only, or text-only, and whether in real time, messages for interacting in quasi-real time (such as a text chat or texting functionality), or messages to be cached for later retrieval (such as an email or answering machine functionality).

In some implementations, the system incorporates a behavior modification reward program; for example, the system has access to a financial API that triggers automatic money transfers to a patient or automatic purchases of gift cards or other goods for a patient. A patient who completes tasks, cooperates with treatment, establishes a pattern of participating fully for a certain number of weeks in a row, and/or shows improvement on various metrics is rewarded by a money transfer, check, gift card, or equivalent, automatically generated and sent to a bank account or address associated with the patient.

Analysis of Individual Behavior Both on a Personal Level and in Contrast to a Collective Data

In some embodiments, a deep learning AI configuration may be used to compare and contrast baseline personal input data including but not limited to text inputs from a variety of sources (e.g., journal entries, emails, personal notes entered into an electronic platform); vocal intonations and cadences (e.g., tone of voice, speed of talking, etc. including indications of emotional variance); login activity (e.g., time of logins, frequency of login activity, length of time logged into platform); assessment answers to health risk questionnaires and other activities; and other methods of data capture not discussed herein. Additionally, individuals may be asked to self-identify events they believe would be correlated to subsequent treatment events to start as baseline markers (the accuracy of which can be tested against with subsequent correlations between the possible presence of any of these and later treatment events).

All individuals may be measured on a variety of domains for a baseline performance, with metrics captured in both a database unique to them (i.e., a personal database), and a database with all metrics for the associated population (i.e., a collective database). Each domain is compared to larger data sets to determine any recognized patterns associated with subsequent treatment events (e.g., improvement, worsening prognosis, return to use, suicide event, etc.) to identify and categorize risk associated with each domain in order to best inform treatment. Correlations established between baseline data points and subsequent treatment events can be used to inform future care.

Continued monitoring of data points throughout the treatment arc will continue to add data points where variances within personal data base as well as any variances associated with treatment events in collective database will initiate a cascade decision process determining whether changes in treatment are indicated to account for potential risk or positive outcomes based on past correlations.

Continued collection and recording of subsequent treatment events and outcomes associated with variances are noted in personal and collective data bases. Deep learning AI configurations continue to explore potential correlations between data points (e.g., vocal intonation, login activity, etc.) and treatment events to identify any potential new correlations or indicators, as well as to strengthen or weaken previously established correlations.

Throughout the whole process, a semi-supervised processed is used wherein clinical judgement is applied on behalf of the clinician overseeing the process and their discretion indicates whether AI recommendations are acted upon in informing treatment changes.

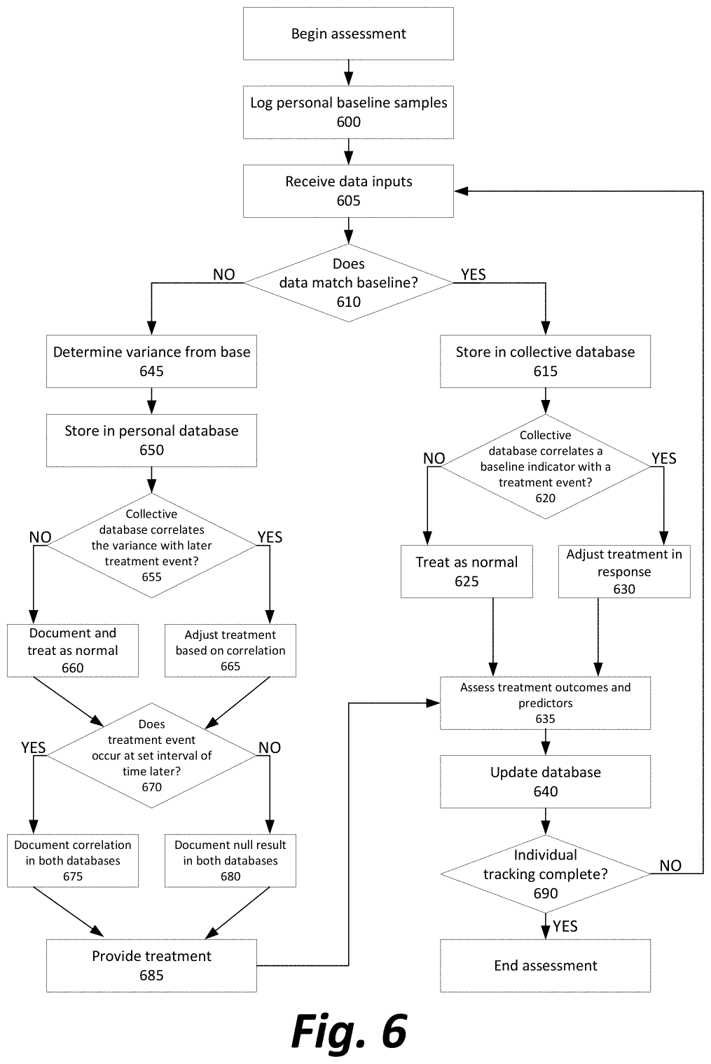

FIG. 6 depicts one example of method for collecting baseline data inputs and comparing it to a personalize and collective database to generate treatment recommendations.

The individual provides a baseline sample (Step 600) of their text (including but not limited to inputs through journal entries, input assessments, baseline vocal samples, and other metrics). Considerations are made at this point based on demographic, native language, and other personalized characteristics to ensure use of the processes outlined herein are appropriate for the individual. These baseline metrics are recorded in a database unique to the individual [600].

Subsequent data is received (Step 605) throughout the course of treatment in various forms and from various entry points. The data received is compared to the initial personal baseline to identify variances (Step 610). This process determines next course of action by identifying whether variances at a significant threshold have been identified. Threshold levels will be set and modified based on updated analytics informed by the collective database (Step 615) suggestive of correlations with subsequent treatment events. Queries are run identifying whether there are any correlations/matches between initial baseline features and larger population indicators of subsequent treatment events (Step 620) to inform recommendations for treatment adaptation (if any). If there are no significant correlations (Step 625), then no special treatment recommendations are made; however, if there are correlations (Step 630) then these are highlighted so treatment accommodates can be made in accordance with likelihood of subsequent treatment event (e.g., positive outcome, negative predictive outcome, etc.). The effectiveness of these changes as well as captured data elements compared to treatment outcomes and subsequent treatment events at set time intervals are processed through a semi-supervised deep learning correlation-based assessment process to inform weighted correlations and significance to best inform future practice (Step 635). The results of these computations are then used to update the personal and collective data bases with indicators and weighted threshold levels used to trigger recommendations for treatment interventions (Step 640).

If initial data received from the individual does differ significantly from their personal baseline, then this information is captured in the technology platform (Step 645) and updated in the personal database (Step 650), which originally stored the baseline samples. These variances are checked against thresholds outlined from the collective database to note whether variances meet threshold level established indicating a change in treatment approach is recommended based on correlations with subsequent treatment events as common in the larger data population (Step 655). If these variances do not meet the threshold, then these variances are still documented in the personal and collective databases in order to reference against later in instances with subsequent treatment events (Step 660) with no suggestions made for adjustment to treatment made by this process. If the input does meet threshold levels as informed by a contrast with the collective database, then the system provides recommendations based on correlations with subsequent treatment events in order to proactively help inform treatment based on predictive methodologies (Step 665). The results of these actions (either treatment recommendations made or not made) are then evaluated (Step 670) to inform whether that decision was the proper course of action taken based on contrast with subsequent treatment events at set time intervals (e.g., 24 hours later, one week later, one month later, etc.). The subsequent effects of whether action was taken or not taken and its correlation to subsequent treatment events (Steps 675 and 680 respectively) are used then inform and update both personal and collective databases and inform treatment that is provided (Step 685). The results of these actions are then assessed at time intervals to determine any changes of thresholds to inform future treatment (Step 635) and to update both the personal and collective databases (Step 640) to complete this iteration of the process (Step 690), which then initiates a process loop until treatment completion and post discharge follow up, with continuing monitoring resulting in repeating the loop (back to Step 610), or ending the process completely.

Applications Beyond an Individual Patient

Using the data gathered from a number of patients leads to at least two advantages.

First, incoming patients may have a better baseline of comparison for risk when the system is able to see the outcomes of numerous other patients who had had similar values for one or more measured factors.

Second, particular treatment teams, centers, companies, or protocols may be assessed for effectiveness based on their statistics for success comparative to others for similar patients. Insurers assessing whether to work with or continue supporting a particular provider of medical services or method of providing medical services will have greater data for optimizing patient outcomes and preventing the cost associated with relapse after treatment.

1. A system for monitoring and adjusting the psychiatric care of a human patient, comprising:
a server, long-term memory storage, and a mobile computing device,
wherein the server and the mobile computing device comprise instructions that, when executed by a processor of the server, establish a feedback loop by causing:
the mobile computing device to transmit data from one or more sensors of the mobile computing device representing the human patient's actions while in possession of the mobile computing device;
the server to receive the transmitted data;
the server to retrieve from the long-term memory storage both baseline data related to the human patient and collective data related to a number of other human patients;
the server to determine that the transmitted data either differs from the baseline data in a statistically significant manner or matches elements of the collective data in a manner that suggests a heightened risk of harm to the human patient;
the server to transmit, to the mobile computing device, instructions causing a change in functionality of the mobile computing device; and
the mobile computing device to implement the change in functionality upon receipt of the instructions.

2. The system of claim 1, wherein the one or more sensors comprise a GPS sensor, wherein a difference of the transmitted data from the baseline data is the human patient's location in a new city, and wherein the change in functionality causes the mobile computing device to display directions to the human user to a location in the new city.

3. The system of claim 1, wherein the one or more sensors comprise an accelerometer, wherein a determination is made that the transmitted data indicates the human patient is awake at a time that the human patient has been advised to sleep, and wherein the change in functionality dims the screen of the mobile computing device or causes it to be unable to display entertainment to the human patient.

4. A system for monitoring and adjusting the psychiatric care of a human patient, comprising:
a server and long-term memory storage,
wherein the server comprises instructions that, when executed by a processor of the server, establish a feedback loop by causing:
the server to receive data transmitted from one or more sensors of a mobile computing device representing the human patient's actions while in possession of the mobile computing device;
the server to retrieve from the long-term memory storage both baseline data related to the human patient and collective data related to a number of other human patients;
the server to determine that the transmitted data either differs from the baseline data in a statistically significant manner or matches elements of the collective data in a manner that suggests a heightened risk of harm to the human patient;
the server to transmit, to the mobile computing device, instructions causing a change in functionality of the mobile computing device.

5. The system of claim 4, wherein the one or more sensors comprise a GPS sensor, wherein a difference of the transmitted data from the baseline data is the human patient's location in a new city, and wherein the change in functionality causes the mobile computing device to display directions to the human user to a location in the new city.

6. The system of claim 5, wherein the location in the new city is the site of a support meeting, and wherein the server receives from the mobile computing device a verification that the human patient has attended a support meeting at the location.

7. The system of claim 5, wherein the location in the new city is a medical clinic, and wherein the server receives from the computing device associated with the clinic a verification that the human patient has obtained a service at the clinic.

8. The system of claim 4, wherein the one or more sensors comprise an accelerometer, wherein a determination is made that the transmitted data indicates the human patient is awake at a time that the human patient has been advised to sleep, and wherein the change in functionality dims the screen of the mobile computing device or causes it to be unable to display entertainment to the human patient.

9. The system of claim 4, wherein the one or more sensors comprise a microphone, wherein a determination is made that the transmitted data indicates vocal qualities of statement made by the human patient, and wherein the change in functionality causes display of a message or video to the human patient.

10. The system of claim 4, wherein the one or more sensors comprise a camera, wherein a determination is made that the transmitted data indicates that a prescription medication has been obtained by the human patient, and wherein the change in functionality causes display of a message or video to the human patient.

11. A computer-implemented method for monitoring and adjusting the psychiatric care of a human patient, comprising:
establishing a feedback loop that comprises:
receiving data transmitted from one or more sensors of a mobile computing device representing the human patient's actions while in possession of the mobile computing device;
retrieving from long-term memory storage both baseline data related to the human patient and collective data related to a number of other human patients;
determining that the transmitted data either differs from the baseline data in a statistically significant manner or matches elements of the collective data in a manner that suggests a heightened risk of harm to the human patient; and
transmitting, to the mobile computing device, instructions causing a change in functionality of the mobile computing device.

12. The method of claim 11, wherein the one or more sensors comprise a GPS sensor, wherein a difference of the transmitted data from the baseline data is the human patient's location in a new city, and wherein the change in functionality causes the mobile computing device to display directions to the human user to a location in the new city.

13. The method of claim 12, wherein the location in the new city is the site of a support meeting, and wherein the server receives from the mobile computing device a verification that the human patient has attended a support meeting at the location.

14. The method of claim 12, wherein the location in the new city is a medical clinic, and wherein the server receives from the computing device associated with the clinic a verification that the human patient has obtained a service at the clinic.

15. The method of claim 11, wherein the one or more sensors comprise an accelerometer, wherein a determination is made that the transmitted data indicates the human patient is awake at a time that the human patient has been advised to sleep, and wherein the change in functionality dims the screen of the mobile computing device or causes it to be unable to display entertainment to the human patient.

16. The method of claim 11, wherein the one or more sensors comprise a microphone, wherein a determination is made that the transmitted data indicates vocal qualities of statement made by the human patient, and wherein the change in functionality causes display of a message or video to the human patient.

17. The method of claim 11, wherein the determination that suggests a heightened risk of harm to the human patient is based at least in part on a random forest classification algorithm.

18. The method of claim 11, wherein the determination that suggests a heightened risk of harm to the human patient is additionally based at least in part on subjective data received from one of more human users in communication with the human patient.

19. The method of claim 18, wherein the subjective data received from one of more human users is provided via a graphical user interface.
